(19) United States
(12) Patent Application Publication
(10) Pub. No.: US 2007/0196871 A1
(43) Pub. Date: Aug. 23, 2007

(54) Forensic Test for Human Semen

(75) Inventor: Karl Reich, Hillside, IL (US)

Correspondence Address: BAKER & MCKENZIE LLP, Pennzoil Place, South Tower, 711 Louisiana, Suite 3400, HOUSTON, TX, 77002-2716, US

(73) Assignee: INDEPENDENT FORENSICS, INC., Hillside, IL (US)

(21) Appl. No.: 11/678,057

(22) Filed: Feb. 22, 2007

Related U.S. Application Data
(60) Provisional application No. 60/775,821, filed on Feb. 22, 2006.

(51) Int. Cl. G01N 33/567; G01N 33/558

(52) U.S. Cl. 435/7.2; 436/514

(57) ABSTRACT

Lateral flow immunochromatographic strip tests for the detection of human semen, method of detecting human semen, and methods of manufacturing ICS tests for the detection of human semen are described

### PRIOR RELATED APPLICATION

This application claims priority to U.S. Provisional Application Ser. No. 60/775,821, filed Feb. 22, 2006, which is incorporated by reference herein in its entirety.

### FEDERALLY SPONSORED RESEARCH STATEMENT

Not applicable.

### REFERENCE TO MICROFICHE APPENDIX

Not applicable.

### FIELD OF THE INVENTION

This invention relates generally to the field of forensics and specifically to the definitive detection of human semen from forensic samples derived from crime scenes, sexual assault evidence kits, and other relevant samples. The invention uses an immmuno-based lateral flow or dip-stick test with specificity for semen that is previously undescribed. Current forensic methods to identify semen are often presumptive—assuming a stain is semen based on type or location—and may identify target antigens not specific to semen.

In contrast, the invented test uses two antibodies configured as individual capture and detection components and is the first such test that uses two antibodies for the species specific identification of human semen that excludes false detection of other body fluids and semen of other species.

### BACKGROUND OF THE INVENTION

Currently, semen testing kits are used for two different purposes by two separate communities. Police departments and forensic labs use sophisticated kits for rape analysis and consumers use a commercially available variant called a ‘cheater kit’ for detecting infidelity. Both types of kits measure Prostate Specific Antigen (PSA, also known as P30) or Acid Phosphatase activity (AP).

Unfortunately, acid phosphatase activity is not confined to semen or prostatic tissue. In fact, vaginal secretions can contain identical lysozymes and phosphatases. Immunoassays for PSA are specific for PSA (also known as P30), but substantial levels of P30 have also been found in amniotic fluid and breast milk, as well as in male blood, serum and urine. In addition, female urine has been shown to cross-react with commercial P30 tests, and low levels of P30 may be detected in vaginal fluid. Thus, crime labs that rely on AP or PSA technology often obtain false positive results, leading to analysis and investment in samples that lack human semen.

The economic impact of sexual assault in the U.S. is considerable: the average rapist commits between eight and 13 rapes before being convicted. Only a small percentage of sexual assaults are actually reported, even fewer are processed for further analysis, and a small percentage of processed cases are prosecuted, thus allowing many rapists to commit more crimes. Finally about 200,000 rape samples are analyzed each year in the U.S. Back-calculating on the serial losses at each step, a conservative estimate indicates that at least 1,000,000 sexual assaults are never analyzed, processed or prosecuted. A fast, accurate and inexpensive test to help crime laboratories correctly identify samples for more sophisticated DNA analysis would be of enormous benefit.

Antibody tests for detecting semenogelin are being investigated and used by a number of groups (Sato, 2001; Yoshida, 2003; Sato, 2004). Sato, et al. have developed a dot-blot-immunoassay for semen identification using a polyclonal antibody against semenogelin (Sato, 2001). Yoshida, et al. have quantified seminal plasma motility inhibitor (SPMI) and semenogelin in human seminal plasma (Yoshida, 2003). However, these groups employ a single monoclonal antibody and a polyclonal antibody, and polyclonal antibodies have been shown to be less sensitive for semenogelin than PSA and AP tests (Lawson, 1998). Although these tests provide an assay of semenogelin, it is surprising to note that third parties have shown the dual monoclonal antibody tests described herein are 3-fold more sensitive than these prior art tests. A test that will detect and identify small amounts of sample, yet be specific and highly sensitive for only human semen is required.

### SUMMARY OF THE INVENTION

The invention is generally directed to immuno-based tests for human seminal plasma motility inhibitor, also known as “semenogelin.” The function of semenogelin resides in its ability to form a gel in pre-ejaculated semen and its ability to be degraded by PSA/P30 and thus liquefy the semen some time after ejaculation. This is direct contrast to many animal species where newly ejaculated semen is liquid and then forms a gel. The protein is unique to human semen, and sufficiently differs between species to be an ideal antigen for a human semen immuno-based test.

As used herein, human semenogelin means the protein described at GenBank Acc # NP_{—}002998, NP_{—}937782, and NP_{—}002999, incorporated by reference. Semenogelin may also include semenogelin 1 (SEMG1), semenogelin 1a (SEMG1a), semenogelin 1b (SEMG1b), semenogelin 2 (SEMG2), seminal basic protein, alpha-inhibin-31, and alpha-inhibin-92, depending upon expression and proteolytic cleavage. All of these forms, and immunogeneic peptides thereof, are considered to be semenogelin herein.

The invention provides the first truly specific confirmatory test for human seminal fluid. The test strips do not cross react with other body fluids, including saliva, blood, urine, or vaginal secretions, nor do they cross react with seminal fluids from other species. Further, the test strips are sensitive, detecting as little as 1 μl of human seminal fluid. Additionally, test strip results correlate with quantitative forensic results obtained for short tandem repeat (STR) sequences from the Y chromosome (Y-STR) in profile intensity (except from vasectomized or low sperm count males). Further, the test strip can be employed in 10 minutes, providing a quick, sensitive and specific assay with an extended shelf life.

In one embodiment, the invention involves the use of two monoclonal antibodies that each recognize distinct, non-overlapping epitopes unique to human semenogelin. The antibodies are configured in a lateral flow immunochromatographic strip (ICS) test such that the capture antibody is immobilized on a porous substrate at a defined position. The detection antibody, which is labeled for visualization, is placed on the substrate at the origin. Samples are deposited onto the substrate at the origin, where any semenogelin in the sample will form a semenogelin-detection antibody complex. The complex migrates along the porous substrate with the capillary action of the buffer and is then captured by the immobilized capture antibody. This complex becomes visible to the naked eye as it is immobilized and concentrated on the test line by being bound by the capture antibody.

Detection antibodies are monoclonal antibodies or recombinant monoclonal antibodies, SEG1, SEG2, SEG3, or other antibodies with the requisite characteristics. Detection antibodies are labeled for detection by a variety of known methods. Generally they are conjugated with colloidal gold, streptavidin, biotin, microspheres, peroxidase, horse radish peroxidase (HRP), streptavidin-labeled HRP, phosphatase, alkaline phosphatase (AP), chromogenic labels, fluorescent labels, chemiluminescent labels, phosphorescent labels and the like.

Immobilization or capture antibodies are monoclonal antibodies, recombinant antibodies, SEG1, SEG2, or SEG3, or other antibodies with the requisite characteristics. Immobilization antibodies may be adsorped, crosslinked, chemically conjugated, or bound by a variety of linker molecules to affix the protein to the substrate. Many proteins will adsorp rapidly to nitrocellulose, PVDF, or other substrates without further processing.

In one aspect, the present invention comprises a test for the presence of human semen or seminal fluid that consists of two monoclonal antibodies which each bind to unique non-overlapping epitopes present on human semenogelin. The monoclonal antibodies do not cross-react with the semen of other species, nor with other body fluids.

In another aspect, the invention is directed to a lateral flow test strip wherein one of the monoclonal antibodies, the capture antibody, is immobilized on a solid substrate, bead or surface and used with the detection antibody, which is labeled or derivatized and the pair are used to specifically detect human semenogelin.

In another embodiment, the invention is directed to monoclonal antibodies SEG1, SEG2, or SEG3, Each of these antibodies binds to distinct non-overlapping epitopes, but does not bind to semen from other species, nor to non-semen body fluids.

The ICS tests can be assembled in a cassette with one or more strips in a cassette for parallel tests. This is particularly useful for a cassette containing a negative (−) control without antigen and/or a positive (+) control with a known concentration of human semenogelin. ICS tests may also be provided in a kit with instructions, sample swabs, swab wetting solution, sample tubes, sample tube holder, scissors, sample extraction buffer, sample running buffer, transfer pipet, or completed ICS test documentation envelope, and the like.

### BRIEF DESCRIPTION OF THE DRAWINGS

FIG. 1: Lateral Flow Immunochromatographic Strip Test for Detection of Human Semen.

FIG. 2: Test Line Intensity Comparison Chart. Control and test lines are quantified a by comparison with the Intensity Score Sheet.

### DETAILED DESCRIPTION OF THE INVENTION

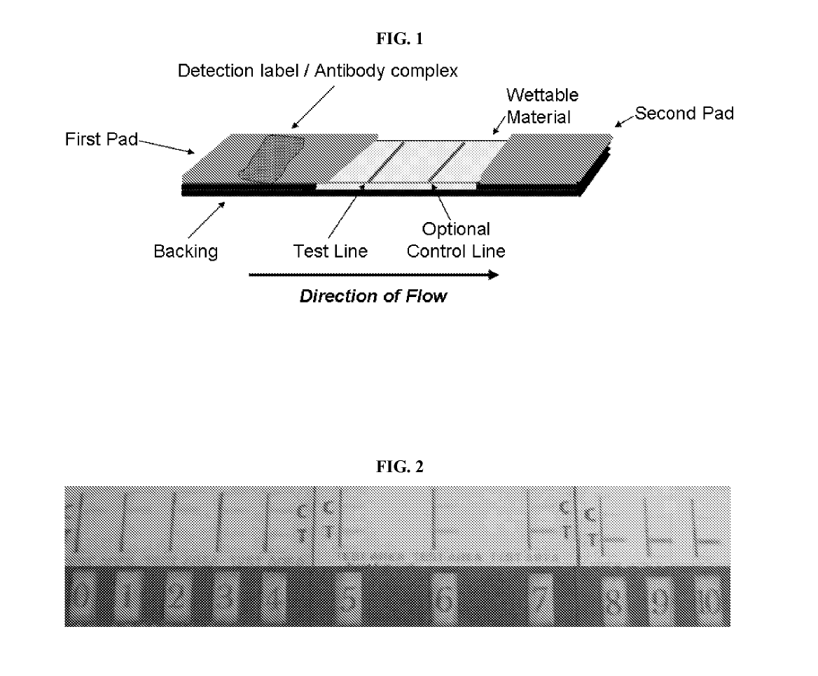

FIG. 1 depicts a preferred design of the lateral flow test strip. The three components of this preferred test strip are the conjugate pad, the membrane, and the wick (sometimes referred to herein as the absorbent pad). The conjugate pad can be made from any absorbent or porous material such as glass-fiber paper, rayon, cotton or polyester. The membrane can be made from nitrocellulose or other suitable material with or without a non-absorbent backing. The wick can be made from any absorbent material such as glass-fiber paper, filter paper, rayon, cotton or polyester, and the like.

An additional component, a sample pad, is sometimes used in addition to the conjugate pad and functions as secondary filter for complex extracts and is placed above the conjugate pad and can be made from any absorbent or porous material such as glass-fiber paper, filter paper, rayon, cotton or polyester, and the like.

The conjugate pad contains antibodies labeled for visualization. Any detectable label can be used with the detection antibody. The membrane is appropriately configured to contain both a ‘control line’ and a ‘test line’. The test line contains anti-semenogelin capture antibodies, responsible for immobilizing and concentrating the semenogelin-antibody complex as the sample travels past the test line. The control line should be placed at a position on the membrane beyond the test line such the sample passes by the test line first and then encounters the control line. The control line provides a visual readout of the correct assembly of the strip, the proper function of the strip and contains an anti-antibody source (e.g., anti-mouse IgG antibody) that captures and concentrates any remaining un-bound detection antibody that was not captured by the test line, indicating that the sample has indeed chromatographed past the test line. The wick soaks up the sample to prevent backflow during the detection time window.

The test line on FIG. 1 illustrates a positive reaction for human semenogelin. The control line indicates the detection antibody has been properly released from the conjugate pad and has travelled past the test line, confirming the proper functioning of the test strip.

The dip-stick can be provided in kit form. The kits of the invention may also include instructions for use, extraction buffers, running buffers, labels, methods and containers for collecting samples, envelopes with tamper evident tape for sample storage, transfer pipettes, pre-filled tubes, single use scissors or punches, and similar useful components to make use of the strip test in the field or at a crime scene.

We have described herein one preferred dip-stick embodiment, but there are many variations on the dip stick design. Other designs are possible including annular ring designs, blot cards, and test sticks (immersed in sample/conjugate antibody solution). Further, we have described a dip-stick that uses a conjugate pad for the sample origin, but the origin may also be directly on the membrane or any porous substrate. Fluid migration may be monitored by control line intensity, dye front, or color changes when damp.

### EXAMPLE 1

### Antibodies

The antibodies used herein were obtained from Riitta Koistinen, Department of Clinical Chemistry, Helsinki University Central Hospital, Biomedicum Helsinki, 4.floor, P.O. Box 700, Haartmaninkatu 8, 0029 HUS, Helsinki, FINLAND. However, monoclonal antibody preparation is well known in the art, and additional antibodies can be made according to well understood procedures.

| Antibody | MAB Designation |
|---|---|
| SEG1 | F91-11E6 |
| SEG2 | F91-1D6 |
| SEG3 | F92-10G7 |

These antibodies were tested (data not shown) and each can bind to semenogelin at the same time, indicating that the epitopes recognized by these monoclonal antibodies do not overlap. Additional tests (see below) established that the antibodies are specific for human sperm, and do not cross react with other body fluids.

For labeling, 200 micrograms of each antibody (at 10 microgram/ml concentration) was added to 20 ml colloidal gold, pH 8.2, and gently rocked for 60 minutes after which 2.2 ml of 10% BSA (for 1% BSA final concentration) was added and gently rocked for 1 hour.

### EXAMPLE 2

### Dip-Stick Manufacture

Glass fiber conjugate pad material was cut into strips 300 mm long by 22 mm wide. To make the conjugate pad more hydrophilic, the strips were pretreated by immersion in a solution containing sodium tetraborate (0.2%), Bovine Serum Albumin (BSA, 3%), polyvinylpyrrolidone (PVP, 1%), sucrose (0.1%), and Triton X-100 (0.25%) for 10 minutes (5 minutes on each side). The treated pads were blotted dry and dried in an oven at 37° C. for one hour. The dried conjugate pads were stored in an airtight and moisture resistant foil pouch containing desiccant.

Next the relevant antibodies were applied to the strip components with all possible combinations of detection and capture antibodies, as follows:

SEG1-gold, SEG2-gold and SEG3-gold conjugates were spun down at 16,000 G for 25 minutes with no brake at 4 degrees. Supernatant was carefully removed and 1.8 ml of passive gold diluent (0.07% sodium phosphate and 0.1% BSA) was added for an approximate 10-fold dilution. If needed, the volume of conjugate was adjusted to obtain optimal OD of ˜10.

20% w/v sucrose and 5% w/v trehalose was added to 1 ml SEG1-gold, SEG2-gold and SEG3-gold conjugates. These detection antibodies were each dispensed onto a pretreated conjugate pad at 10 μl/cm using an airjet QUANTI™ Dispenser. After dispensing the conjugates onto the pads, the conjugate pads were dried at 37° C. for 1 hour.

SEG1, SEG2, and SEG3 (the capture antibodies) were dispensed onto a nitrocellulose membrane (at the position of the test line) using a BIODOT QUANTI™ dispenser set at 1 μl/cm. Antibodies were concentrated or diluted to a final concentration of 1.5 mg/ml. Antibodies to be tested as capture antibodies were deposited on the test line using the BIODOT QUANTI™ dispenser. Goat anti-mouse IgG antibody was dispensed onto the same membrane at a location 4 mm above the test line hence making the goat anti-mouse IgG antibody the control line. The BIODOT QUANTI™ dispenser can dispense both the control and test line simultaneously. The conjugate pads were similarly prepared using the gold conjugated antibodies.

Components were then assembled using a vacuum assisted laminator with pre-cut dies designed to precisely hold membrane, pad and wick in the correct orientation and with sufficient overlap to make complete strips. Individual components were placed in the laminator in reverse order with the backing card placed on lid (again with the help of pre-cut dies), the adhesive on the backing card revealed, and the laminator closed to adhere the backing card to the remaining strip test components. The laminator was then opened and the assembled card removed. Assembled cards were cut into 3-5 mm sections using a dedicated step motor driven guillotine cutter. The resulting strips were stored over desiccant until use or assembled into appropriate cassette cases.

For test experiments, single strips were placed in glass tubes containing 100 μl of the specified test solution with the conjugate side to the bottom of the glass tube and the liquid traveled up the strip by capillary action. The reaction was stopped by removing the strips from the glass tubes, laying the strips on a paper towel, and removing the conjugate pad with forceps. The intensity of the test and control lines were quantified using the AMERICAN BIO MEDICA ™ Corporation Intensity Score Sheet after 15 minutes.

A variety of test materials were used to determine the optimal combination of membrane, conjugate pad and blocking solutions with each antibody combination. These include different nitrocellulose membranes, different conjugate pad materials, different mixtures of detergents and buffers for blocking. The use of sample pads were also be tested depending on the sample origin.

Various combinations of antibody were tested, per the table below. The intensity of the test strip line was scored by visual comparison against an Intensity Score standard (see e.g., FIG. 2), that consisted of a series of graded reddish lines, from faint to strong, where the displayed line was given an intensity score. The operator compared the test line of the strip test against the score sheet, and recorded the observed intensity. This method helped to minimize operator variance and provided quantitative data for validation.

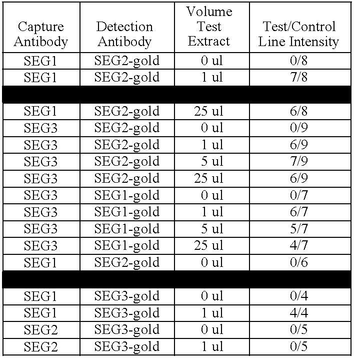

These experiments demonstrated that the best combination was SEG1 test line and SEG2-gold conjugate—a result that could not be predicted a priori.

### EXAMPLE 3

### Quantification of Results

For sensitivity studies, we prepared a sample whereby 50 μl semen was deposited on a cotton swab and allowed to air-dry. The tip of the cotton swab was cut off, placed in a 1.5 ml microfuge tube, and extracted in 1 ml PBS for 1 hour at room temperature. Assuming 100% extraction efficiency, each μl of extract will contain approximately 50 nl of semen (concentration of 50 nl saliva/μl extract).

A high dose “Hook effect” refers to the false negative seen with ICS tests when very high levels of target are present in the tested sample. Under these conditions, unbound semenogelin antigen reaches the test line before the semenogelin-antibody-gold complex, thereby preoccupying the test line with non-labeled semenogelin and resulting in a false negative result.

To assess the threshold level of the high dose Hook effect, we also tested increasing amounts of the semen extract. Ten dilutions of semen extract (as well as a sample lacking semen extract for a negative control) were tested in two different experiments with the semenogelin strip tests by adding the indicated semen extract to a final volume of 100 μl with PBS+or TBS+running buffer and placing the 100 μl solution in the sample window of the cassette. The control and test lines in the strip test window were quantified after 10 minutes by comparison with the Intensity Score Sheet described above.

| Vol of Semen | | Equivalent Vol | | PBS + Buffer | TBS + Buffer |
|---|---|---|---|---|---|
| Ext | | of Semen | | (T/C) | (T/C) |
| 0 | | 0 | | 0/8 | 0/7 |
| 1 | μl @ 1:25 | 2 | nl | 0/8 | 0/7 |
| 1 | μl @ 1:10 | 5 | nl | 1/8 | 1/7 |
| 1 | μl @ 1:5 | 10 | nl | 3/8 | 2/7 |
| 1 | μl | 50 | nl | 7/8 | 6/7 |
| 2 | μl | 100 | nl | 8/8 | 7/7 |
| 5 | μl | 250 | nl | 9/8 | 7/7 |
| 10 | μl | 500 | nl | 8/8 | 6/6 |
| 25 | μl | 1.25 | μl | 7/7 | 5/6 |
| 50 | μl | 2.5 | μl | 6/6 | 4/6 |
| 100 | μl | 5 | μl | 5/6 | 4/5 |

In PBS+buffer the highest band intensity was seen with 1, 2, 5, and 10 μl of semen extract after 10 minutes. 1 μl at 1:5 dilution (5 nl semen) was clearly positive after 10 minutes. This indicates that the ICS test for semen can detect as little as 10 nl human semen. The signal gradually decreased with 25, 50, and 100 μl semen extract, indicative of a high dose Hook effect when analyzing 1.25, 2.5, and 5 μl of human semen, respectively.

In TBS+Buffer, the sensitivity was slightly lower than in PBS+Buffer, but there was less background signal too. The highest band intensity was seen with 1, 2, and 5 μl semen extract after 10 minutes. The signal gradually decreased with 10, 25, 50, and 100 μl extract after 10 minutes.

To test the threshold the Hook effect, we extracted semen into a smaller volume of PBS in order to concentrate the components of the semen. Assuming 100% extraction, each μl of extract from a 400 μl extraction should contain 125 nl of semen. The results were as follows:

| Vol of Semen Ext | | Equivalent Vol of Semen | | Results (T/C) |
|---|---|---|---|---|
| 0 | | 0 | | 0/7 |
| 5 | μl (1 ml ext) | 250 | nl | 7/7 |
| 1 | μl (400 μl ext) | 125 | nl | 6/6 |
| 5 | μl (400 μl ext) | 625 | nl | 4/6 |
| 25 | μl (400 μl ext) | 3.125 | μl | 3/6 |
| 50 | μl (400 μl ext) | 6.25 | μl | 2/5 |
| 100 | μl (400 μl ext) | 12.5 | μl | 1/5 |
| ˜50 | μl (200 μl ext) | 50 | μl | 2/5 |

These experiments indicate that a false negative result is possible when analyzing greater than 3 μl semen in a sample. To address this, we recommend a dilution of the sample 1:10-1:20 if a low signal is observed using the semenogelin strip test. For example, diluting a sample that originally contains approximately 3-5 μl semen by a factor of 20 will result in measuring semen in the range of approximately 150-250 nl semen, an amount that produces a strong signal using the semenogelin strip test (data not shown).

In conclusion, the optimal signal with the semenogelin strip tests was seen when analyzing between 50 and 500 nl semen, and the high dose Hook effect was evident when testing between 1.25 and 5 μl semen, although no true false negatives (i.e., no signal whatsoever) were seen. Our choice of TBS+as the running buffer for Semenogelin ICS test for future experiments was predicated on the lower background seen with this running buffer on numerous experiments (data not shown).

### EXAMPLE 4

### Antigen Stability in TBS+

To determine whether semen extracts are stable in TBS+ (pH 8.5) for extended incubation times, semen extracts where incubated for four hours and overnight (16.5 hours) in TBS+ before running on strips. Extracts that were added to running buffer immediately prior to strip application were included for comparison.

| Sample | Incubation | Result at 10 minutes (T/C) |
|---|---|---|
| 5 μl sham extract | none | 0/9 |
| 5 μl semen extract | none | 5/8 |
| 5 μl semen extract | 4 hours | 7/8 |
| 5 μl sham extract | none | 0/8 |
| 5 μl semen extract | none | 6/8 |
| 5 μl semen extract | overnight | 6/8 |

Incubation of semen extract in TBS+ for four hours or overnight at room temperature had no effect on the background or sensitivity of the saliva strip test. Extracts were stable in TBS+ for the Semenogelin ICS test detection.

We also tested a variety of buffers for extraction efficiency, as follows:

| Buffer | Recipe |
|---|---|
| TE | 10 mM Tris, 1 mM EDTA, pH 8.0 |
| PBS | 1.0 mM NaH2PO4, 8.1 mM Na2HPO4, 150 mM NaCl, |
| | pH 7.4 |
| PBS+ | 1.0 mM NaH2PO4, 8.1 mM Na2HPO4, 150 mM NaCl, |
| | 0.01% Tween-20, pH 7.4 |
| TBS | 50 mM Tris, 137 mM NaCl, 2.7 mM KCl, 0.01% |
| | NaN3, pH 8.5 |
| TBS+ | 50 mM Tris, 137 mM NaCl, 2.7 mM KCl, 0.01% |
| | NaN3, 0.01% Tween-20, 0.5% BSA F.V, pH 8.5 |

| | | | Result at 10 |
|---|---|---|---|
| Sample | | Extraction | minutes (T/C) |
| 5 | μl sham extract (swab) | PBS | 0/8 |
| 5 | μl semen extract (swab) | PBS | 7/8 |
| 10 | μl semen extract (punch) | PBS | 7/8 |
| 10 | μl semen extract (punch) | Water | 4/8 |
| 10 | μl semen extract (punch) | TE | 7/8 |
| 10 | μl semen extract (punch) | TBS+ | 7/8 |
| 1 | μl semen extract (punch) | PBS | 2/8 |
| 1 | μl semen extract (punch) | Water | 1/8 |
| 1 | μl semen extract (punch) | TE | 3/8 |
| 1 | μl semen extract (punch) | TBS+ | 4/8 |

The punch extracted with water gave the weakest signal, clearly weaker than the punch extracted in PBS. Punches extracted in TE and TBS+ gave positive results that were indistinguishable from punches extracted with PBS. Low signals were NOT due to High Dose Hook effect, because all samples were retested with a 1:10 dilution and the results correlated with the initial tests.

### EXAMPLE 5

### Body Fluid Specificity

We next tested extract from swabs of various fluid alone or as a mixture to determine body fluid specificity. 50 μl of saliva, urine, semen, or blood were each deposited on separate cotton swabs and allowed to air-dry. The tip of the swab with the cotton batting was cut off using laboratory clean technique and placed in 1.5 ml microcentrifuge tube and extracted into 1 ml PBS for 2 hours at room temperature. Extracts from body fluids were combined with and without semen to evaluate potential cross-reactivity and possible inhibition. Five μl of semen extract was tested in the presence of 25 μl extract from the other three body fluids (saliva, blood & urine), or all three body fluids (25 μl of each extract, blood, saliva & urine) were combined and tested on Semenogelin ICS test. All tested volumes were brought to 100 μl with running buffer. Assuming 100% extraction efficiency, 25 μl of body fluid extract from these samples was approximately 1.25 μl whole body fluid and 4 μl total body fluid.

| | | Result at 10 |
|---|---|---|
| Sample | | minutes (T/C) |
| 5 | μl semen | 9/8 |
| 5 | μl semen plus saliva | 9/8 |
| 5 | μl semen plus urine | 9/8 |
| 5 | μl semen plus blood | 9/8 |
| 5 | μl semen plus 25 μl each saliva, blood and urine | 9/8 |
| 25 | μl saliva | 0/8 |
| 25 | μl urine | 0/8 |
| 25 | μl blood | 0/8 |
| 25 | μl each saliva, blood and urine | 0/8 |
| 5 | μl sham | 0/8 |

The sensitivity of the semenogelin strip test with semen extract alone or in combination other fluids was the same: both scored as ˜9 after 10 minutes. There was no positive signal observed with the other fluids alone. Similar results (not shown) were seen with non-exposed vaginal fluids, breast milk, and non-exposed saliva, although semen could be detected in saliva where there was oral-genital contact. Therefore, we conclude that there was no cross-reactivity of urine, blood, saliva or vaginal fluids and that the sensitivity to semen extract was not affected by the presence of other body fluids.

### EXAMPLE 6

### Species Cross Reactivity Test

To investigate whether semen from species other than human will give a positive result with semenogelin ICS tests, we tested air dried cotton swabs containing 50 μl of semen from various domestic animals. The swabs were extracted in 1.0 ml of PBS and 20 μl and 1 μl (to address high dose Hook effect) of the extract was applied to semen strip tests. Also, since some animal semen samples were stored with an extender (nourishment for sperm and antibiotics), a strip was run with an extract from human semen mixed with extender (50 μl human semen +50 μl extender was applied to a cotton swab which was air dried and extracted with 1 ml of PBS) in order to see if the extender affected results. Positive (50 μl of human semen extracted in 1 ml of PBS) and negative (clean cotton swab extracted in 1 ml of PBS) controls were included for comparison.

| | |
|---|---|
| | Result at 10 |
| Sample (volume of extract) | minutes (T/C) |

| | | |
|---|---|---|
| 5 | μl sham | 0/8 |
| 5 | μl human semen | 6/8 |
| 20 | μl goat | 0/8 |
| 20 | μl sheep | 0/8 |
| 20 | μl pig | 0/8 |
| 20 | μl bull | 0/8 |
| 20 | μl dog | 0/8 |
| 20 | μl horse | 0/8 |
| 20 | μl mouse | 0/8 |
| 20 | μl cat | 0/8 |
| 5 | μl Human semen + Extender | 5/8 |

No cross reaction was present at the test line of any of the strips run with animal semen extracts at either dilution. In addition, semen extender did not interfere with semenogelin ICS test detection. Note: all tests were re-tested at 1:20 dilution to insure that no false negative results were recorded. The re-tests also showed no cross-reaction with the test (data not shown). Therefore, we concluded that the semenogelin test was specific for human semen.

### EXAMPLE 7

### Stability of ICS Test for Semen

We have previously demonstrated that semenogelin ICS test was both specific and sensitive for human semen, but another critical issue is stability of the strip tests in storage. We tested storage of the strip tests at 37° C. for one month, which mimics storage at room temperature for one year. The stability of the semenogelin ICS test strip was not affected by storage at 37° C. for 30 days, as compared to strip tests stored at room temperature (data not shown). The sensitivity of detection was the same under both conditions. Therefore, we concluded that the strip had a shelf life of at least one year.

### EXAMPLE 8

### Detection in Forensic Samples

We clearly established that the semenogelin ICS test could detect semen from a laboratory prepared control sample; but we also demonstrated the ability of the test to detect semen from a variety of samples likely to be encountered in forensic laboratory case work. For example, underwear were swabbed and tested as above, and the results were as follows:

| | |
|---|---|
| Sample | Result at 10 minutes (T/C) |

| | | |
|---|---|---|
| 5 | μl sham | 0/8 |
| 10 | μl underwear swab | 8/8 |
| 10 | μl underwear swab | 8/8 |
| 5 | μl semen extract | 7/8 |

Furthermore, a acid phosphatase test was performed on the underwear stain which was recorded as only “weakly” positive. These results indicated that the test had increased sensitivity as compared to current methods of semen detection.

Similar results were obtain from vaginal swabs where no condom was used for up to two days after coitus. These results were also reflected in DNA-based STR testing whereby DNA was extracted from the samples and analyzed for male DNA using Y-filer. A partial DNA profile was obtained from swabs at day 0, 1, and 2, but no profiles were obtained from swabs on days 3 and beyond. Further testing showed that the presence of menstrual blood did not negatively affect the test.

### EXAMPLE 9

### Test of Exhibit Extraction Procedure

An important issue in any semen detection method, is compatibility with the method used to collect the test sample. Two general approaches are in forensic laboratory practice: cuttings and/or transfer to moistened swab. We directly compared these two methods on a variety of fabric substrates: swabbing the stain with a moistened cotton swab or extracting a cutting of defined size (a 5 mm diameter circle made with a stainless steel Harris punch).

Tester samples were prepared by depositing 50 μl of semen onto the following different types of fabrics: 1) cotton chambray, 2) flannel cotton sheet, 3) cotton twill, 4) cotton denim), 5) nylon lace), 6) nylon knit jersey, and 7) cotton sheet. Each fabric type was sampled using a ddH₂O-moistened cotton swab or by excising a 5 mm diameter circle using a SS Harris punch. Each swab was extracted in 200 μl PBS for 1 hour at room temperature and each 5 mm punch was extracted in 100 μl PBS for 1 hour at room temperature. We tested 20 μl of the swab extract and 10 μl of the 5 mm punch extract with semenogelin ICS test.

The results (not shown) generally indicated that a punch is preferred to a swab, presumably due to superior extraction. Further, swab extraction was clearly fabric dependent. The signal from a swab extract from the nylon lace and nylon knit jersey was high, ˜8 for both fabrics after 10 minutes. However, the signal from swab extracts from all cotton fabrics was low, ranging from ˜1 for cotton denim to ˜4 for flannel cotton sheet.

The extracts were subsequently tested for DNA-STR using Y-FILER™ in order to determine if a correlation between the semenogelin ICS test signal intensity and DNA content could be observed. The results (data not shown) were clear: partial DNA profiles ranging from 10 to 14 loci were observed from the 5 mm punch extracts, while only 4-6 loci were observed from the swab extracts.

Therefore, we concluded that a punch was a better and more efficient sampling method than a swab, particular for highly absorbent fabrics.

### EXAMPLE 10

### Effects of Contraceptives and Lubricants

We next determined whether commercially available consumer contraceptives/or vaginal lubricant interfered with the semenogelin ICS test. 100 μl of semen was mixed with 100 μl of various lubricant or contraceptives. Then, 100 μl of the mixture was pipetted onto a cotton sheet, allowed to air dry, and stored for future use. 50 μl of semen was pipetted onto a different section of the same cotton sheet and analyzed side-by-side as an extraction control. 5 mm punches of the stains were extracted in 100 μl of PBS, and any low samples were re-tested at a 20 fold dilution to account for the Hook effect.

| | Result at 10 |
|---|---|
| Sample | minutes (T/C) |
| sham | 0/8 |
| positive | 8/8 |
| KY ® + semen | 2/8 |
| VCF ® + semen | 5/8 |
| Conceptrol ® + semen | 4/8 |
| Semen alone @ 1:20 | 3/8 |
| Semen alone @ 1:20 | 8/8 |
| Semen from no lubricant condom @ 1:20 | 7/8 |
| Semen from non-spermicidal lubricant condom @ 1:20 | 6/8 |
| Semen from spermicidal lubricant condom @ 1:20 | 7/8 |

Semen was detected in all samples containing semen and no background was observed. Some of the strips gave clear positive results on dilution, suggesting that the originally weak signals were due to the High Dose Hook effect and not interference by the condoms. Users should be aware of High Dose Hook effects and should perform a 1:20 dilution of samples that test negative or weakly positive in order to eliminate the possibility of false negatives.

### EXAMPLE 11

### Sensitivity of Monoclonal Antibodies

The sensitivity of the test strips described herein with 2 monoclonal antibodies was compared to test strips with 1 monoclonal antibody and 1 polyclonal antibody as described in Sato, 2004 by an independent third party. This group determined that the 2 monoclonal antibody test strip was 3-fold more sensitive than the test strip with polyclonal antibodies. Therefore, we concluded that the 2 monoclonal antibody test strip is significantly more sensitive than the prior art method.

We have described elongated dip-stick tests herein for purposes of illustration only. However, other designs are possible including annular ring designs, blot cards, and test sticks (immersed in sample/conjugate antibody solution). Kits described herein are for demonstration purposes and additional components may be added.

### REFERENCES

All references are listed herein for the convenience of the reader. Each is incorporated by reference in its entirety.
1, Berti, et al,. “Expression of seminal vesicle-specific antigen in serum of lung tumor patients.” J Forensic Sci. 50:1114-5 (2005).
2, Sato, et al., “Rapid detection of semenogelin by one-step immunochromatographic assay for semen identification.” J Immunol Methods 287(1-2): 137-45 (2004).
3, Yoshida, et al., “Quantification of seminal plasma motility inhibitor/semenogelin in human seminal plasma.” J Androl (6):878-84 (2003).
4, Sato, et al., “A dot-blot-immunoassay for semen identification using a polyclonal antibody against semenogelin, a powerful seminal marker. ” Forensic Sci Int. 122(1):27-34 (2001).
5, Herr JC and Woodward MP., “An enzyme-linked immunosorbent assay (ELISA) for human semen identification based on a biotinylated monoclonal antibody to a seminal vesicle-specific antigen.” J Forensic Sci. 32(2):346-56 (1987).
6, Keil W, et al. “Evaluation of MHS-5 in detecting seminal fluid in vaginal swabs.” Int J Legal Med. 108(4):186-90 (1996).
7, Lawson, M., et al. “Objective markers of condom failure.” Sex Transm Dis 25:427 (1998).

What is claimed is:

1. A test for human semen,
said test comprising a porous substrate having in order: an origin and a test portion,
wherein a detection antibody comprising a labeled monoclonal anti-human semenogelin antibody is placed at the origin, and a capture antibody comprising a monoclonal anti-human semenogelin antibody is fixed at the test portion,
wherein the detection and capture antibodies do not bind non-semenogelin proteins or to semenogelin from non-human species, but can simultaneously and specifically bind to human semenogelin.

2. The test of claim 1, wherein said porous substrate has a control portion with a control antibody that specifically binds the detection antibody fixed at the control portion.

3. The test of claim 1, wherein said test can detect less than 1 μl human semen in a forensic sample.

4. The test of claim 1, wherein said test is at least 3-fold more sensitive than a test with one or more polyclonal antibodies.

5. The test of claim 1, wherein the capture antibody is SEG1 and the detection antibody is SEG2.

6. The test of claim 1, wherein said test is a lateral flow immunochromatographic strip (ICS) test comprising:
a) a first pad comprising the detection antibody;
b) a wettable material comprising the capture antibody, and
c) a second pad, wherein said wettable material (b) connects said first pad (a) to said second pad and said second pad is an absorbent material.

7. The test of claim 6, wherein said detection antibody is SEG2 and said capture antibody is SEG1.

8. A kit for the detection of human semen, said kit comprising a container having:
a porous substrate having, in order, an origin and a test portion, wherein
a first monoclonal antibody specific for human semenogelin and labeled for detection is reversibly applied to the origin, and
a second monoclonal antibody specific for human semenogelin is immobilized on the test portion,
wherein the first and second antibodies do not bind to non-semenogelin proteins, do not bind to semenogelin of non-human species, but can simultaneously bind to human semenogelin,
b) instructions for use of the kit, and
c) optionally containing one or more of a sample collector, a sample container, an extraction buffer, an intensity comparison chart, sample swabs, swab wetting solution, sample tubes, sample tube holder, scissors, punches, sample extraction buffer, sample running buffer, transfer pipet, test documentation envelope, and a label.

9. The kit of claim 8, wherein said ICS can detect less than 1 μl human semen in a forensic sample.

10. The kit of claim 8, wherein said ICS is at least 3 fold more sensitive than a test card with one or more polyclonal antibodies.

11. The kit of claim 8, wherein said first antibody is SEG1, SEG2, or SEG3.

12. The kit of claim 8, wherein said second antibody is SEG1, SEG2, or SEG3.

13. The kit of claim 8, wherein said porous substrate is a lateral flow immunochromatographic strip (ICS) test comprising:
a) a first pad comprising the first antibody;
b) a membrane comprising the second antibody, and
c) a second pad, wherein said membrane (b) connects said first pad (a) to said second pad wherein said second pad is an absorbent material.

14. The kit of claim 13, wherein said detection antibody is SEG2 and said capture antibody is SEG1.

15. An immunochromatographic strip (ICS) test comprising:
a) a first pad comprising a first monoclonal anti-human semenogelin antibody labeled for detection,
b) a wettable material comprising a second human semenogelin antibody immobilized in a test portion,
c) a second pad, wherein said wettable material (b) connects said first pad (a) to said second pad wherein said second pad is an absorbent material,
wherein the first and second antibodies do not bind to non-semenogelin proteins or to semenogelin from non-human species, but can simultaneously and specifically bind to human semenogelin.

16. The ICS of claim 15, wherein said ICS can detect less than 1 μl human semen in a forensic sample.

17. The ICS of claim 15, wherein said ICS is at least 3 fold more sensitive than a test card with one or more polyclonal antibodies for detection of human semen in a forensic sample.

18. The ICS of claim 15, wherein said first antibody is SEG1, SEG2, or SEG3.

19. The ICS of claim 15, wherein said second antibody is SEG1, SEG2, or SEG3.

20. The ICS of claim 15, wherein said first antibody is SEG2 and said second antibody is SEG1.
